(19) United States
(12) Patent Application Publication
(10) Pub. No.: US 2010/0036164 A1
(43) Pub. Date: Feb. 11, 2010

(54) PREPARATION OF ARMODAFINIL FORM I

(76) Inventors: Srinivas Areveli, Karimnagar (IN); Radha Krishna Singamsetty, Hyderabad (IN); Satish Kumar Vujjni, Hyderabad (IN); Vijaya Kumar Adla, Warangal (IN); Sreenadha Charyulu Kandala, Hyderabad (IN); Ganesh Varanasi, Hyderabad (IN); Subbareddy Peddireddy, Cuddapah (IN); Mohammed Azeezulla Baig, Vijayawada (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/535,789

(22) Filed: Aug. 5, 2009

Related U.S. Application Data
(60) Provisional application No. 61/105,837, filed on Oct. 16, 2008.

(30) Foreign Application Priority Data
Aug. 6, 2008 (IN) 1892/CHE/2008

(51) Int. Cl. C07C 231/00

(52) U.S. Cl. 564/162

(57) ABSTRACT

Preparation of armodafinil crystalline Form I. Also provided is armodafinil having about 30% or more by weight of particles with sizes greater than about 250 μm, and about 70% or less by weight of particles having sizes less than about 250 μm, wherein of the particles having sizes less than about 250 μm, about 50% of them have diameters less than about 50 μm.

Aspects of the present application relate to processes for preparing crystalline Form I of armodafinil.

The sulphur atom in the sulphoxide moiety of the compound known as “modafinil” is found to exhibit optical isomerism, which makes modafinil exist as a mixture of R— and S-enantiomers. The R-enantiomer of modafinil is used as a drug and has a chemical name (−)-2-[(R)-(diphenylmethyl)sulfinyl]acetamide, or 2-[(R)-benzhydrylsulfinyl]acetamide, (hereinafter referred to by the adopted name “armodafinil”) and is represented by structural Formula I.

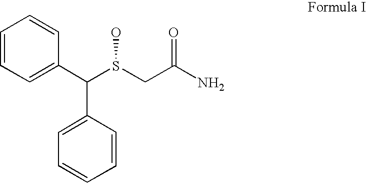

Armodafinil is a wakefulness-promoting agent for oral administration, which is approved for marketing in the USA for improving wakefulness in patients with excessive sleepiness associated with obstructive sleep apnea/hypopnea syndrome, narcolepsy and shift work sleep disorder. Armodafinil is known by its brand name NUVIGIL™ in the various strengths 50, 150 and 250 mg.

U.S. Pat. No. 4,927,855 discloses armodafinil and process for its preparation. It also describes pharmaceutical compositions comprising armodafinil and their use in the treatment of hypersomnia and Alzheimer's disease. The patent also discloses resolution of modafinic acid followed by its conversion to armodafinil. In this process, two additional crystallization steps are performed in order to increase the enantiomeric purity of the R-modafinic acid. As a consequence, the overall yield reported in the patent is rather unsatisfactory. In this patent, the solvent used for final crystallization of armodafinil is ethanol.

International Application Publication No. WO 2007/103221 describes resolution of modafinic acid with α-naphthylethylamine. α-Naphthylethylamine is a costly reagent, which is not desirable for cost-effectiveness of the process.

Resolution of modafinic acid requires the simplest way to prepare armodafinil without the need to use sophisticated equipment, such as preparative HPLC or SMB (simulated moving bed) technology, or expensive and sensitive reagents, such as those generally used in asymmetric synthesis of armodafinil. Even though methods are available for resolution of modafinic acid, there remains a need for more efficient methods with simple conditions that provide high yields and quality.

U.S. Pat. No. 7,132,570 describes polymorphic Form I of armodafinil, a process for its preparation and a pharmaceutical composition comprising crystalline Form I of armodafinil.

International Application Publication No. WO 2007/098273 describes a process for preparation of crystalline Form I of armodafinil by drying crystalline Form C, crystalline Form D, or crystalline Form IV of armodafinil, or by humidifying crystalline Form A of armodafinil, or by grinding or applying pressure to crystalline Form IV of armodafinil.

Over the years, more than 40% of the potential candidates in drug discovery and research have failed to emerge as drugs due to their poor biopharmaceutic properties. Many of these are rejected due to poor solubility characteristics and further development is continued only if the new molecule has some marked advantage over the existing molecules indicated for the similar use.

The most common approach used to address the problem of low solubility is by either reducing the drug's particle size or micronizing the drug to the size of a few microns, which increases the effective exposed surface area. Dosage forms which contain micronized drug particles exhibit enhanced solubility and consequently exhibit an increase in the bioavailability of the drugs. However, technical and economical problems may sometimes arise due to micronization. For example, highly micronized drug particles can possess poor flow properties and an increased chance of re-agglomeration during processing. In some cases, re-agglomeration of micronized drug particles may be so problematic that the basic objective of enhancing the solubility by increasing the effective surface area may be unmet.

U.S. Pat. No. RE37516 discloses a pharmaceutical composition comprising a substantially homogeneous mixture of modafinil particles, wherein at least about 95% of the cumulative total of modafinil particles in the composition have a diameter less than about 200 μm.

U.S. Pat. No. 7,115,281 discloses an oral dosage form comprising about 7% to 25% by weight of modafinil particles having diameters greater than 220 μm, and about 93% to 75% by weight of modafinil particles having diameters less than 220 μm, wherein about 90% of the particles having diameters size less than 220 μm are further characterized in that they have diameters less than about 41 μm, and about 50% of the particles having diameters size less tan 220 μm are further characterized in that they have diameters less than about 21 μm

There is a need to provide crystalline forms of active substances such as armodafinil with a desired particle sizes in an industrially simple and readily feasible way with high yields.

### SUMMARY

An aspect of the present application provides processes for preparation of crystalline Form I of armodafinil, an embodiment including:

a) providing a solution of armodafinil in a suitable solvent;

b) causing precipitation of a solid; and

c) optionally, drying the solid.

Another aspect of the present application provides improved processes for the preparation of armodafinil, an embodiment comprising:

a) condensation of thiourea with diphenylmethanol, in the presence of an acid in a suitable solvent, to obtain an acid addition salt of S-benzhydryl-thiouronium which may be further converted to benzhydryl thioacetic acid;

b) oxidation of benzhydryl thioacetic acid to obtain (±)-benzhydrylsulfinyl acetic acid;

c) treating (±)-benzhydrylsulfinyl acetic acid with L-(−)-α-methylbenzylamine in a suitable solvent and isolating (−)-α-methylbenzylamine (−)-benzhydrylsulfinyl acetic acid diastereomeric salt;

d) converting (−)-α-methylbenzylamine (−)-benzhydrylsulfinyl acetic acid diastereomeric salt into (−)-benzhydrylsulfinyl acetic acid in a suitable solvent in the presence of an acid, and isolating the (−)-benzhydrylsulfinyl acetic acid;

e) esterification of (−)-benzhydrylsulfinyl acetic acid to a C₁-C₄ lower alkyl (−)-benzhydrylsulfinyl acetate;

f) amidation of C₁-C₄ lower alkyl (−)-benzhydrylsulfinyl acetate obtained in step e) and isolating armodafinil.

An aspect of the present application provides armodafinil having about 30% or more by weight of armodafinil particles having sizes greater than about 250 μm, and about 70% or less by weight of armodafinil particles having sizes less than about 250 μm, wherein of the particles having sizes less than about 250 μm, about 50% of them have sizes less than about 50 μm.

An aspect of the present application provides armodafinil Form I having about 30% or more by weight of armodafinil particles having sizes greater than about 250 μm, and about 70% or less by weight of armodafinil particles having sizes less than about 250 μm, wherein of the particles having sizes less than about 250 μm, about 50% of them have sizes less than about 50 μm.

An aspect of the present application provides pharmaceutical compositions comprising armodafinil prepared according to a process of the present application and at least one pharmaceutically acceptable excipient.

Another aspect of the present application provides pharmaceutical compositions comprising armodafinil Form I prepared according to a process of the present application and at least one pharmaceutically acceptable excipient.

An aspect of the present application provides pharmaceutical compositions comprising armodafinil having about 30% or more by weight of armodafinil particles having sizes greater than about 250 μm, and about 70% or less by weight of armodafinil particles having sizes less than about 250 μm, wherein of the particles having sizes less than about 250 μm, about 50% of them have sizes less than about 50 μm, and at least one pharmaceutically acceptable excipient.

Another aspect of the present application provides pharmaceutical compositions comprising armodafinil Form I having about 30% or more by weight of armodafinil particles having sizes greater than about 250 μm, and about 70% or less by weight of armodafinil particles having sizes less than about 250 μm, wherein of the particles having sizes less than about 250 μm, about 50% of them have sizes less than about 50 μm, and at least one pharmaceutically acceptable excipient.

### BRIEF DESCRIPTION OF THE DRAWINGS

FIG. 1 is an X-ray powder diffraction (“XRPD”) pattern for an illustrative sample of armodafinil Form I, prepared according to Example 2.

FIG. 2 is a differential scanning calorimetry (“DSC”) curve for an illustrative sample of armodafinil Form I, prepared according to Example 2.

FIG. 3 is a thermogravimetric analysis (“TGA”) curve for an illustrative sample of armodafinil Form I, prepared according to Example 2.

### DETAILED DESCRIPTION

An aspect of the present application provides processes for preparation of crystalline Form I of armodafinil, an embodiment including:

a) providing a solution of armodafinil in a suitable solvent;

b) causing precipitation of a solid; and

c) optionally drying the solid.

Armodafinil that may be used for providing a solution in the above process may be prepared, e.g., by a process disclosed in this application, or it may be prepared by any processes known in the art.

The solution of armodafinil may be obtained by dissolving armodafinil in a suitable solvent, or such a solution may be obtained directly from a reaction mixture in which armodafinil is formed.

Suitable solvents include, but are not limited to: alcohols such as methanol, ethanol, 2-butanol and the like; ketones such as acetone, methyl ethyl ketone and the like; halogenated hydrocarbons such as dichloromethane, chloroform, and the like; polar aprotic solvents such as N,N-dimethylformamide, N,N-dimethylacetamide, dimethylsulphoxide, N-methylpyrrolidone, and the like; ethers such as tetrahydrofuran, 1,4-dioxane, and the like; nitriles such as acetonitrile and the like; carboxylic acids such as formic acid, acetic acid and the like; nitromethane; ethylene glycol; and any mixtures thereof.

Suitable temperatures for dissolution of armodafinil in a suitable solvent may range from about 10° C. to about 150° C., or about 20° C. to about 120° C., or about 40° C. to about 100° C., or about 50° C. to about 70° C., or the reflux temperature of the solution, depending on the solvent used.

Armodafinil Form I may be formed from the solution of armodafinil in the solvent by processes known in the art. Various techniques that may be used for forming armodafinil Form I include, but are not limited to, cooling, distillation, evaporation, combining with an anti-solvent, and any other techniques known in the art for the recovery of solids from a solution. The solution may optionally be concentrated by methods such as evaporation or distillation before cooling the solution or combining with an anti-solvent, for solid formation. Also, a small amount of seed crystals of armodafinil Form I can be added to promote crystallization.

“Anti-solvent” as used herein refers to a solvent that reduces the solubility of the solute in the solution. Suitable anti-solvents include, but are not limited to: water; alcohols such as isopropyl alcohol, n-butanol and the like; ketones such as methyl isobutyl ketone and the like; aromatic hydrocarbons such as toluene, xylenes and the like; esters such as ethyl acetate, and the like; ethers such as diethyl ether, diisopropyl ether, methyl t-butyl ether, anisole and the like; aliphatic hydrocarbons such as n-pentane, hexanes, n-heptane, cyclohexane, and the like; and any mixtures thereof.

Suitable temperatures to which the solution may be cooled are lower than the solution formation temperatures, such as less than about 100° C., or less than about 80° C., or less than about 60° C., or less than about 40° C., or less than about 30° C., or less than about 20° C., or less than about 10° C., or less than about −10° C., or less than about −20° C., or any other suitable temperatures to cause precipitation of the solid. Suitable times for cooling the solution of armodafinil may vary considerably, such as from about 2 to about 8 hours, or longer, depending on the desired extent of recovery. However, the exact cooling temperatures and times required for complete crystallization can be readily determined by a person skilled in the art and will also depend on parameters such as concentration and temperature of the solution or slurry. Stirring or other alternate methods such as shaking, agitation and the like, that mix the contents may also be employed for precipitation to occur.

Before or after cooling the solution of armodafinil, the solution may be seeded with a small amount of crystals of armodafinil Form I to induce crystallization of armodafinil Form I. The seed crystals of armodafinil may be prepared by processes known in the art or by a process of the present application.

The formed crystals may be recovered by conventional methods including decantation, centrifugation, gravity filtration, suction filtration or any other technique known in the art for the recovery of solids. The crystals thus isolated may carry some amount of occluded mother liquor and thus have higher than desired levels of impurities. If desired, these crystals may be washed with a solvent or a mixture of solvents to wash out the impurities.

The recovered solid may optionally be further dried. Drying may be carried out in a tray dryer, vacuum oven, air oven, fluidized bed dryer, spin flash dryer, flash dryer and the like. The drying may be carried out at temperatures less than about 150° C., or less than about 120° C., or less than about 100° C., or less than about 80° C., or less than about 60° C., or any other suitable temperature as long as armodafinil Form I is not degraded in quality, at atmospheric pressure or under a reduced pressure. The drying may be carried out for any desired time until the required purity is achieved. For example, it may vary from about 4 to about 8 hours, or longer.

An aspect of the present application provides improved processes for the preparation of Armodafinil, an embodiment including:

a) condensation of thiourea with diphenylmethanol, in the presence of an acid in a suitable solvent, to obtain an acid addition salt of S-benzhydryl-thiouronium which may be further converted to benzhydryl thioacetic acid;

b) oxidation of benzhydryl thioacetic acid to obtain (±)-benzhydrylsulfinyl acetic acid;

c) treating (±)-benzhydrylsulfinyl acetic acid with L-(−)-α-methylbenzylamine in a solvent and isolating (−)-α-methylbenzylamine (−)-benzhydrylsulfinyl acetic acid diastereomeric salt;

d) converting (−)-α-methylbenzylamine (−)benzhydrylsulfinyl acetic acid diastereomeric salt in to (−)-benzhydrylsulfinyl acetic acid in a solvent in the presence of an acid and isolating (−)-benzhydrylsulfinyl acetic acid;

e) esterification of (−)-benzhydrylsulfinyl acetic acid to a C₁-C₄ lower alkyl (−)-benzhydrylsulfinyl acetate; and

f) amidation of C₁-C₄ lower alkyl (−)-benzhydrylsulfinyl acetate obtained in step e) to form armodafinil.

Step a) involves reacting thiourea with diphenylmethanol in the presence of an acid in a suitable solvent to give an acid addition salt of S-benzhydryl-thiouronium.

The acids used herein include, but are not limited to, inorganic acids, organic acids or acid-based resins. Suitable acids include, but are not limited to: inorganic acids such as hydrochloric acid, hydrobromic acid, hydroiodic acid, hydrofluoric acid, sulfuric acid, nitric acid, phosphoric acid, polyphosphoric acid and the like; and organic acids such as acetic acid, oxalic acid, tartaric acid, n-propionic acid, isopropionic acid, n-butyric acid, isobutyric acid, and the like; or any suitable acid-functional resins.

Suitable solvents which may be used in step a) include, but are not limited to: alcohols such as methanol, ethanol, isopropyl alcohol, n-propanol, n-butanol, 2-butanol and the like; ketones such as acetone, ethyl methyl ketone, methyl isobutyl ketone, and the like; halogenated hydrocarbons such as dichloromethane, ethylene dichloride, chloroform, and the like; hydrocarbon solvents such as toluene, xylene, n-hexane, n-heptane, cyclohexane and the like; esters such as ethyl acetate, n-propyl acetate, n-butyl acetate, t-butyl acetate, and the like; ethers such as diethyl ether, diisopropyl ether, methyl t-butyl ether, tetrahydrofuran, dioxane, and the like; polar aprotic solvents such as N,N-dimethylformamide, N,N-dimethylacetamide, dimethylsulphoxide, sulpholane, N-methylpyrrolidone, and the like; nitriles such as acetonitrile, propionitrile, and the like; water; and and mixtures thereof.

The reaction may be carried out at elevated temperatures, ranging from about 10° C. to about 150° C., or about 20° C. to about 120° C., or about 40° C. to about 100° C., or about 60° C. to about 90° C., or about the reflux temperature depending on the solvent used, or any other suitable temperatures.

The formed solid may be recovered by conventional methods including decantation, centrifugation, gravity filtration, suction filtration or other techniques known in the art for the recovery of solids. The resulting solid may be further dried. Drying may be suitably carried out using a tray dryer, vacuum oven, air oven, fluidized bed dryer, spin flash dryer, flash dryer and the like. The drying may be carried out at temperatures from about 25° C. to about 150° C., at atmospheric pressure or under reduced pressure, and in the presence or absence of an inert atmosphere such as nitrogen, argon, neon, or helium.

The acid addition salt of S-benzhydrylthiouronium obtained from the above reaction may be further reacted with chloroacetic acid in the presence of a suitable base to give benzhydryl thioacetic acid.

Useful bases include, but are not limited to organic bases, inorganic bases, and ion exchange resins. Suitable organic bases include, but are not limited to: aliphatic amines such as triethylamine, tributylamine, N-methylmorpholine, N,N-diisopropyl-ethylamine, N-methylpyrrolidine and the like; and aromatic amines such as pyridine, N,N-dimethylaminopyridine, and the like. Suitable inorganic bases include, but are not limited to, alkali metal carbonates, alkali metal bicarbonates, alkali metal hydroxides, and the like. Suitable alkali metal carbonates include, but are not limited to, sodium carbonate, potassium carbonate, and the like. Suitable alkali metal bicarbonates include, but are not limited to, sodium bicarbonate, potassium bicarbonate and the like. Suitable alkali metal hydroxides include, but are not limited to, sodium hydroxide, potassium hydroxide, and the like. Suitable ion exchange resins include, but are not limited to, resins bound to ions such as sodium, potassium, lithium, calcium, magnesium and the like.

The reaction may be carried out at temperatures ranging from about −10° C. to about 150° C., or about 10° C. to about 120° C., or about 20° C. to about 100° C., or any other suitable temperatures.

The suitable times for completion of the reaction depend on the temperature and other conditions and may generally range from about 10 minutes to about 8 hours, or about 1 hour to about 7 hours, or about 1½ hours to about 5 hours, or any other suitable times.

After completion of the reaction, the reaction mass may be decomposed by addition of water and the pH of the reaction mixture may be adjusted to less than about 7 using acids such as hydrochloric acid, hydrobromic acid, sulphuric acid, nitric acid, acetic acid, propionic acid, butyric acid, valaric acid and the like, or an ion exchange resin. The formed solid can be recovered by conventional methods including decantation, centrifugation, gravity filtration, suction filtration or other techniques known in the art for the recovery of solids. The resulting solid may be optionally further dried. Drying may be suitably carried out using a tray dryer, vacuum oven, air oven, fluidized bed dryer, spin flash dryer, flash dryer and the like. The drying may be carried out at temperatures less than about 120° C., or less than 100° C., or less than about 60° C., or less than about 40° C., or any other suitable temperatures, at atmospheric pressure or under reduced pressure, and in the presence or absence of an inert atmosphere such as nitrogen, argon, neon, or helium. The drying may be carried out for any desired time periods to achieve the desired product purity, such as times ranging from about 1 to about 15 hours, or longer.

Step b) involves oxidation of benzhydrylthioacetic acid to obtain (±)-benzhydrylsulfinyl acetic acid.

The oxidation of benzhydrylthioacetic acid may be carried out in a suitable solvent in the presence of a suitable acid.

Useful oxidizing agents include, but are not limited to, hydrogen peroxide, alkali metal hypohalites such as sodium hypochlorite, sodium hypobromite, potassium hypochlorite, potassium hypobromite, and the like, m-chloroperbenzoic acid, peracetic acid, cumene hydroperoxide, sodium perborate, ε-phalimidoper-hexanoic acid, and the like.

Suitable acids include, but are not limited to, inorganic acids, organic acids, and ion exchange resins. Suitable inorganic acids include, but are not limited to, hydrochloric acid, hydrobromic acid, hydroiodic acid, hydrofluoric acid, sulfuric acid, nitric acid, phosphoric acid, polyphosphoric acid, and the like. Suitable organic acids include, but are not limited to, acetic acid, oxalic acid, tartaric acid, n-propionic acid, isopropanoic acid, n-butyric acid, isobutyric acid, and the like. Suitable ion exchange resins include, but are not limited to, phosphoric acid resins, sulphonic acid resins, p-toluene sulphonic acid resins, and the like.

Suitable solvents that may be used in step b) include, but are not limited to: alcohols such as methanol, ethanol, isopropyl alcohol, n-propanol, n-butanol, 2-butanol and the like; ketones such as acetone, ethyl methyl ketone, methyl isobutyl ketone, and the like; halogenated hydrocarbons such as chloroform, dichloromethane, ethylene dichloride, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, dioxane, tetrahydrofuran, and the like; polar aprotic solvents such as dimethylsulphoxide, N,N-dimethylformamide, N,N-dimethylacetamide, N-methylpyrrolidone, sulpholane, and the like; nitriles such as acetonitrile, propionitrile, and the like; water; and any mixtures thereof.

The oxidation may be carried out at temperatures ranging from about −20° C. to about 100° C., or about 0° C. to about 50° C., or about 10° C. to about 40° C., or any other suitable temperatures.

The formed solid can be recovered by methods including decantation, centrifugation, gravity filtration, suction filtration, or other techniques known in the art for the recovery of solids.

The obtained solid may be washed with a suitable solvent. Suitable solvents which may be used for washing include, but are not limited to: alcohols such as methanol, ethanol, isopropyl alcohol, n-propanol, n-butanol, 2-butanol, and the like; ketones such as acetone, ethyl methyl ketone, methyl isobutyl ketone, and the like; halogenated hydrocarbons such as chloroform, dichloromethane, ethylene dichloride, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, tetrahydrofuran, dioxane, and the like; polar aprotic solvents such as N,N-dimethylformamide, N,N-dimethylacetamide, dimethylsulphoxide, sulpholane, N-methylpyrrolidone, and the like; nitriles such as acetonitrile, propionitrile, and the like; water; and any mixtures thereof.

The resulting solid may optionally be further dried. Drying may be suitably carried out using a tray dryer, vacuum oven, air oven, fluidized bed dryer, spin flash dryer, flash dryer and the like. The drying may be carried out at temperatures less than about 140° C., or less than about 100° C., or less than about 60° C., or less than about 40° C., at atmospheric pressure or under reduced pressure, in the presence or absence of an inert atmosphere such as nitrogen, argon, neon, or helium. The drying may be carried out for any desired time periods to achieve the desired product purity, such as times ranging from about 1 to about 15 hours, or longer.

Step c) involves optical resolution of racemic modafinic acid comprising treating (±)-benzhydrylsulfinylacetic acid with L-(−)-α-methylbenzylamine in a suitable solvent, and isolating (−)-α-methylbenzylamine (−)-benzhydrylsulfinyl acetic acid diastereomeric salt.

Suitable solvents for use in step c) include, but are not limited to: water; alcohols such as methanol, ethanol, isopropyl alcohol, n-propanol, n-butanol, 2-butanol, and the like; ketones such as acetone, ethyl methyl ketone, methyl isobutyl ketone, and the like; halogenated hydrocarbons such as chloroform, dichloromethane, ethylene dichloride, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, dioxane, tetrahydrofuran, and the like; polar aprotic solvents such as dimethylsulphoxide, N,N-dimethylformamide, N,N-dimethylacetamide, N-methylpyrrolidone, sulpholane, and the like; nitriles such as acetonitrile, propionitrile, and the like; and any mixtures thereof.

The resolution in step c) may be carried out at temperatures ranging from about −20° C. to about 120° C., or about 0° C. to about 100° C., or about 20° C. to about 80° C., or any other suitable temperatures.

Isolation of (−)-α-methylbenzylamine (−)-benzhydrylsulfinyl acetic acid diastereomeric salt may be affected by crystallization. The crystallization may be effected by conventional techniques including cooling, evaporating solvent, concentrating the reaction mass, adding an anti-solvent and the like. The suitable temperatures for crystallization may range from about −20° C. to about 100° C., or about −10° C. to about 60° C., or about 0° C. to about 40° C. Suitable times for crystallization may range from about 10 minutes to about 90 minutes, or longer. However, the exact temperatures and times required for complete crystallization may be readily determined by a person skilled in the art and will also depend on parameters such as concentration and temperature of the solution or slurry. Stirring or other alternate methods such as shaking, agitation and the like, that mix the contents may also be employed for crystallization.

The crystallized diastereomeric salt may be recovered by conventional methods including decantation, centrifugation, gravity filtration, suction filtration or any other technique known in the art for the recovery of solids. The recovered crystals may be optionally further dried. Drying may be suitably carried out using a tray dryer, vacuum oven, air oven, fluidized bed dryer, spin flash dryer, flash dryer and the like. The drying may be carried out at temperatures less than about 130° C., or less than about 100° C., or less than about 60° C., or less than about 40° C., at atmospheric pressure or under reduced pressure, and in the presence or absence of an inert atmosphere such as nitrogen, argon, neon, or helium. The drying may be carried out for any desired time periods to achieve the desired product purity, and the times may range from about 1 to about 15 hours, or longer.

Step d) involves converting (−)-α-methylbenzylamine (−)-benzhydrylsulfinyl acetic acid diastereomeric salt in to (−)-benzhydrylsulfinyl acetic acid in a suitable solvent in the presence of an acid, and isolating the (−)-benzhydrylsulfinyl acetic acid.

Suitable solvents thath may be used in step d) include, but are not limited to: water; alcohols such as methanol, ethanol, isopropyl alcohol, n-propanol, n-butanol, 2-butanol, and the like; ketones such as acetone, ethyl methyl ketone, methyl isobutyl ketone, and the like; halogenated hydrocarbons such as dichloromethane, ethylene dichloride, chloroform, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, tetrahydrofuran, dioxane, and the like; polar aprotic solvents such as N,N-dimethylformamide, N,N-dimethylacetamide, dimethylsulphoxide, sulpholane, N-methylpyrrolidone, and the like; nitriles such as acetonitrile, propionitrile, and the like; and any mixtures thereof.

The conversion may be carried out at temperatures ranging from about −20° C. to about 100° C., or about −10° C. to about 90° C., or about 0° C. to about 60° C., or about 10° C. to about 40° C., or any other suitable temperatures.

The pH of the reaction mass may be adjusted to less than about 7, or less than about 5, or less than about 3, or less than about 2, or less than about 1, using a suitable acid. Suitable acids include inorganic acids, organic acids, and ion exchange resins. Suitable inorganic acids include, but are not limited to, hydrochloric acid, hydrobromic acid, hydroiodic acid, hydrofluoric acid, sulfuric acid, nitric acid, phosphoric acid, polyphosphoric acid, and the like. Suitable organic acids include, but are not limited to, acetic acid, oxalic acid, tartaric acid, n-propionic acid, isopropanoic acid, n-butyric acid, isobutyric acid, and the like. Suitable ion exchange resins include, but are not limited to, phosphoric acid resins, sulphonic acid resins, p-toluene sulphonic acid resins, and the like.

The (−)-benzhydrylsulfinyl acetic acid may be isolated from the reaction mass by conventional techniques known in the art including cooling, evaporating solvent, concentrating the reaction mass, adding an anti-solvent, and the like. Suitable temperatures for the precipitation range from about −20° C. to about 100° C., or about −10° C. to about 60° C., or about 0° C. to about 40° C. Suitable times for precipitation range from about 10 minutes to about 90 minutes, or longer. However, the exact temperature and time required for complete crystallization may be readily determined by a person skilled in the art and will also depend on parameters such as concentration and temperature of the solution or slurry. Stirring or other alternate methods such as shaking, agitation and the like, that mix the contents may also be employed for precipitation.

The precipitated solid may be recovered using conventional techniques including decantation, centrifugation, gravity filtration, suction filtration, and other techniques known in the art for the recovery of solids. The recovered solid may be optionally further dried. Drying may be suitably carried out using a tray dryer, vacuum oven, air oven, fluidized bed dryer, spin flash dryer, flash dryer and the like. The drying may be carried out at temperatures less than about 160° C., or less than about 100° C., or less than about 60° C., or less than about 40° C., at atmospheric pressure or under reduced pressure, and in the presence or absence of an inert atmosphere such as nitrogen, argon, neon, or helium. The drying may be carried out for any desired time periods to achieve the desired product purity, and the times may range from about 1 to about 15 hours, or longer.

Step e) involves esterification of (−)-benzhydrylsulfinyl acetic acid to a C₁-C₄ lower alkyl (−)-benzhydrylsulfinyl acetate.

Alcohols for forming C₁-C₄ lower alkyl (−)-benzhydrylsulfinyl acetates include but are not limited to methyl, ethyl, n-propyl, isopropyl, n-butyl, isobutyl, and neobutyl alcohols, and the like. The reagents that may be used for esterification include but are not limited to methyl sulfate, dimethyl sulfate, methyl iodide, ethyl iodide, propyl iodide, and butyl iodide, optionally in the presence of a base.

Useful bases include organic bases, inorganic bases, and ion exchange resins. Suitable organic bases include, but are not limited to: aliphatic amines such as triethylamine, tributylamine, N-methylmorpholine, N,N-diisopropyl-ethylamine, N-methylpyrrolidone and the like; and aromatic amines such as pyridine, N,N-dimethylaminopyridine, and the like. Suitable inorganic bases include but are not limited to alkali metal carbonates, alkali metal bicarbonates, alkali metal hydroxides, and the like. Suitable alkali metal carbonates include, but are not limited to, sodium carbonate, potassium carbonate and the like. Suitable alkali metal bicarbonates include, but are not limited to, sodium bicarbonate, potassium bicarbonate and the like. Suitable alkali metal hydroxides include, but are not limited to, sodium hydroxide, potassium hydroxide and the like. Suitable ion exchange resins include, but are not limited to, resins bound to ions such as sodium, potassium, lithium, calcium, magnesium and the like.

Alternatively, (−)-benzhydrylsulfinyl acetic acid may be reacted with a C₁-C₄ alcohol in the presence of an acid. Suitable acids include inorganic acids, organic acids, and ion exchange resins. Suitable inorganic acids include, but are not limited to, hydrochloric acid, hydrobromic acid, hydroiodic acid, hydrofluoric acid, sulfuric acid, nitric acid, phosphoric acid, polyphosphoric acid, and the like. Suitable organic acids include, but are not limited to, acetic acid, oxalic acid, tartaric acid, n-propionic acid, isopropanoic acid, n-butyric acid, isobutyric acid, and the like. Suitable ion exchange resins include, but are not limited to, phosphoric acid resins, sulphonic acid resins, p-toluene sulphonic acid resins, and the like.

Alternatively, (−)-benzhydrylsulfinyl acetic acid may be converted to an acid halide, which may be subsequently reacted with a C₁-C₄ alcohol to get the desired C₁-C₄ lower alkyl (−)-benzhydrylsulfinyl acetate.

Suitable solvents that may be used in step e) include, but are not limited to: alcohols such as methanol, ethanol, isopropyl alcohol, n-propanol, n-butanol, 2-butanol and the like; ketones such as acetone, ethyl methyl ketone, methyl isobutyl ketone, and the like; halogenated hydrocarbons such as dichloromethane, ethylene dichloride, chloroform, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, tetrahydrofuran, dioxane, and the like; polar aprotic solvents such as N,N-dimethylformamide, N,N-dimethylacetamide, dimethylsulphoxide, sulpholane, N-methylpyrrolidone, and the like; nitriles such as acetonitrile, propionitrile, and the like; water; and any mixtures thereof.

The formed ester may be isolated from the reaction mixture, or the solution containing the said ester that is obtained in step e) may be used as-is, for further processing.

The ester may be isolated from the reaction mixture by conventional techniques including cooling, evaporating solvent, concentrating the reaction mass, adding an anti-solvent, and the like. Suitable temperatures for the isolation may range from about −20° C. to about 100° C., or about −10° C. to about 60° C., or about 0° C. to about 40° C. Suitable times for precipitation may range from about 10 minutes to about 90 minutes, or longer. However, the exact temperatures and times required for complete isolation may be readily determined by a person skilled in the art and will also depend on parameters such as concentration and temperature of the solution or slurry. Stirring or other alternate methods such as shaking, agitation and the like, that mix the contents may also be employed for precipitation.

The precipitated solid that is obtained after isolation may be recovered by conventional techniques known in the art including decantation, centrifugation, gravity filtration, suction filtration, or other techniques known in the art for the recovery of solids. The recovered solid may be optionally further dried. Drying may be suitably carried out using a tray dryer, vacuum oven, air oven, fluidized bed dryer, spin flash dryer, flash dryer and the like. The drying may be carried out at temperatures less than about 100° C., or less than about 60° C., or less than about 40° C., at atmospheric pressure or under reduced pressure, and in the presence or absence of an inert atmosphere such as nitrogen, argon, neon, or helium. The drying may be carried out for any desired time periods to achieve the desired product purity, and the times may range from about 1 to about 15 hours, or longer.

Step f) involves amidation of a C₁-C₄ lower alkyl (−)-benzhydrylsulfinyl acetate obtained in step e), and isolating armodafinil.

The amidation reaction may be carried out in a suitable solvent using ammonia gas at atmospheric pressure or under positive pressure, ammonium hydroxide solution, another source of ammonia such as ammonium carbonate or ammonium acetate, and the like.

Suitable solvents for use in step f) include, but are not limited to: water; alcohols such as methanol, ethanol, isopropyl alcohol, n-propanol, n-butanol, 2-butanol, and the like; ketones such as acetone, ethyl methyl ketone, methyl isobutyl ketone, and the like; halogenated hydrocarbons such as chloroform, dichloromethane, ethylene dichloride, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, dioxane, tetrahydrofuran, and the like; polar aprotic solvents such as dimethylsulphoxide, N,N-dimethylformamide, N,N-dimethylacetamide, N-methylpyrrolidone, sulpholane, and the like; nitriles such as acetonitrile, propionitrile, and the like; and any mixtures thereof.

The amidation may be carried out at temperatures ranging from about −20° C. to about 100° C., or about −10° C. to about 80° C., or about 0° C. to about 60° C., or about 10° C. to about 40° C., or any other suitable temperatures.

Armodafinil may be precipitated from the reaction mixture using conventional techniques known in the art including cooling, evaporating solvent, concentrating the reaction mass, adding an anti-solvent, and the like. The suitable temperatures for the isolation may range from about −20° C. to about 100° C., or about −10° C. to about 60° C., or about 0° C. to about 40° C. Suitable times for precipitation may range from about 10 minutes to about 90 minutes, or longer. However, the exact temperatures and times required for complete isolation may be readily determined by a person skilled in the art and will also depend on parameters such as concentration and temperature of the solution or slurry. Stirring or other alternate methods such as shaking, agitation and the like, that mix the contents may also be employed for precipitation.

The precipitated solid that is obtained may be recovered by conventional techniques including decantation, centrifugation, gravity filtration, suction filtration, and other techniques known in the art for the recovery of solids. The recovered solid may be optionally further dried. Drying may be suitably carried out using a tray dryer, vacuum oven, air oven, fluidized bed dryer, spin flash dryer, flash dryer and the like. The drying may be carried out at temperatures less than about 150° C., or less than about 100° C., or less than about 60° C., or less than about 40° C., at atmospheric pressure or under reduced pressure, and in the presence or absence of an inert atmosphere such as nitrogen, argon, neon, or helium. The drying may be carried out for any desired time periods to achieve the desired product purity, and the times may range from about 1 to about 15 hours, or longer.

The obtained solid may be further recrystallized from a suitable solvent and the crystallized solid may be further dried as described above. Suitable solvents which may be used for recrystallization include, but are not limited to: water; alcohols such as methanol, ethanol, isopropyl alcohol, n-propanol, n-butanol, 2-butanol, and the like; ketones such as acetone, ethyl methyl ketone, methyl isobutyl ketone, and the like; halogenated hydrocarbons such as dichloromethane, ethylene dichloride, chloroform, and the like; hydrocarbons such as toluene, xylene, n-hexane, n-heptane, cyclohexane, and the like; esters such as ethyl acetate, n-propyl acetate, n-butyl acetate, t-butyl acetate, and the like; ethers such as diethyl ether, diisopropyl ether, methyl t-butyl ether, tetrahydrofuran, dioxane, and the like; polar aprotic solvents such as N,N-dimethylformamide, N,N-dimethylacetamide, dimethylsulphoxide, sulpholane, N-methylpyrrolidone, and the like; nitriles such as acetonitrile, propionitrile, and the like; and any mixtures thereof.

The crystallized solid may be recovered using conventional techniques including decantation, centrifugation, gravity filtration, suction filtration and other techniques known in the art for the recovery of solids. The recovered solid may be optionally further dried. Drying may be suitably carried out using a tray dryer, vacuum oven, air oven, fluidized bed dryer, spin flash dryer, flash dryer and the like. The drying may be carried out at temperatures less than about 150° C., or less than about 120° C., or less than about 100° C., or less than about 80° C., or less than about 60° C., or less than about 40° C., or any other suitable temperatures as long as armodafinil is not degraded in quality, at atmospheric pressure or under reduced pressure, and in the presence or absence of an inert atmosphere such as nitrogen, argon, neon, or helium. The drying may be carried out for any desired times until the required purity is achieved. It may vary from about 4 to about 8 hours, or longer.

The resulting armodafinil may be optionally converted to armodafinil Form I, by following a process of the present application.

An aspect of the present application provides armodafinil or armodafinil Form I having about 30% or more by weight of armodafinil particles with sizes greater than about 250 μm, and about 70% or less by weight of armodafinil particles having sizes less than about 250 μm, wherein, of the particles having sizes less than about 250 μm, about 50% of them have diameters less than about 50 μm.

An aspect of the present application provides pharmaceutical compositions comprising armodafinil or armodafinil Form I that is prepared according to a process of the present application, and at least one pharmaceutically acceptable excipient.

An aspect of the present application provides pharmaceutical compositions comprising armodafinil or armodafinil Form I having about 30% or more by weight of armodafinil particles with sizes greater than about 250 μm, and about 70% or less by weight of armodafinil particles having sizes less than about 250 μm, wherein, of the particles having diameters lass than about 250 μm, about 50% of them have sizes less than about 50 μm, and at least one pharmaceutically acceptable excipient.

In an embodiment, armodafinil having coarse particle sizes may be subjected to particle size reduction using any of the techniques known in the art, such as milling using millers including ball, roller, hammer mills and jet mills, grinding, stirring a slurry of armodafinil in a solvent using high rotations per minute, under suitable conditions to get any desired particle sizes of armodafinil.

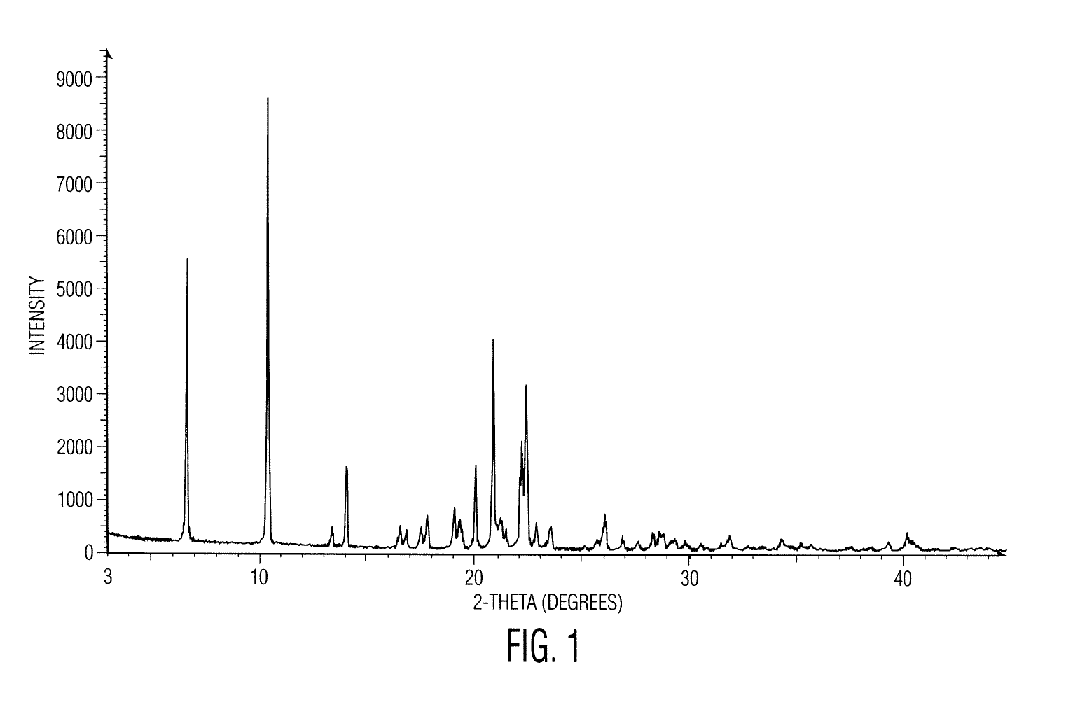

Armodafinil Form I as prepared herein may be characterized by a powder X-ray diffraction pattern (PXRD) having peaks located at approximately 6.6, 10.4, 14.0, 20.0, 20.8, 22.2 and 22.4, ±0.2 degrees 2 theta. Armodafinil Form I may be characterized by its PXRD pattern having peak locations substantially in accordance with the pattern of FIG. 1.

Powder X-ray diffraction analyses reported herein were carried out using copper Kα radiation, and the results were obtained using a Bruker AXS D8 Advance Powder X-ray Diffractometer.

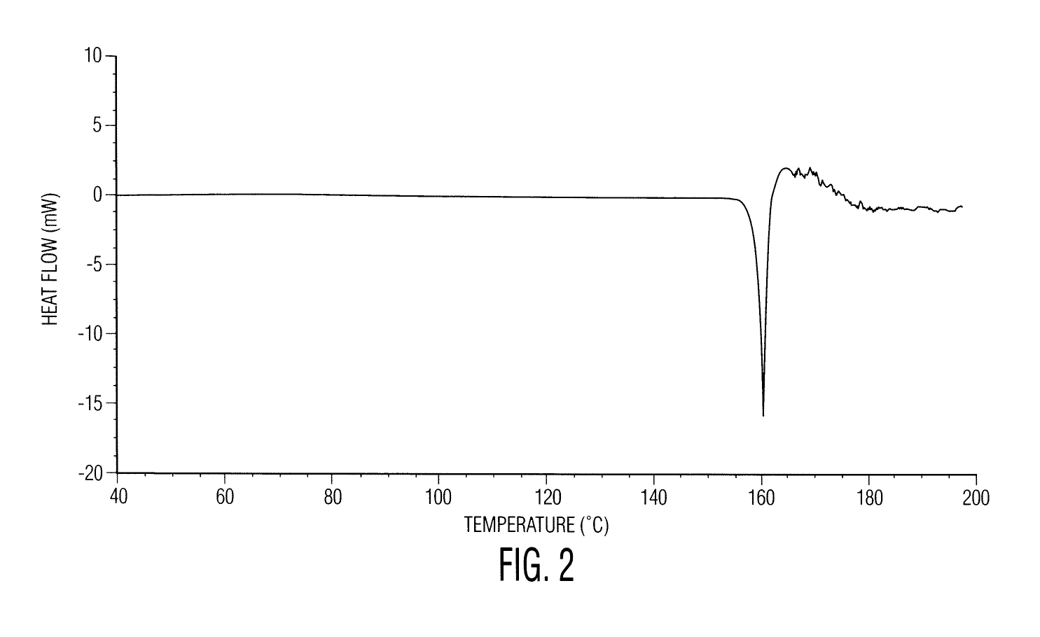

Armodafinil Form I as prepared herein may also be characterized by its differential scanning calorimetry (DSC) curve with a peak at about 160±1° C. Armodafinil Form I may be also characterized by a DSC curve substantially in accordance with FIG. 2.

Differential scanning calorimetric analysis has been carried out using a DSC Q1000 instrument from TA Instruments with a ramp of 5° C./minute. The starting temperature was 40° C. and ending temperature was 200° C.

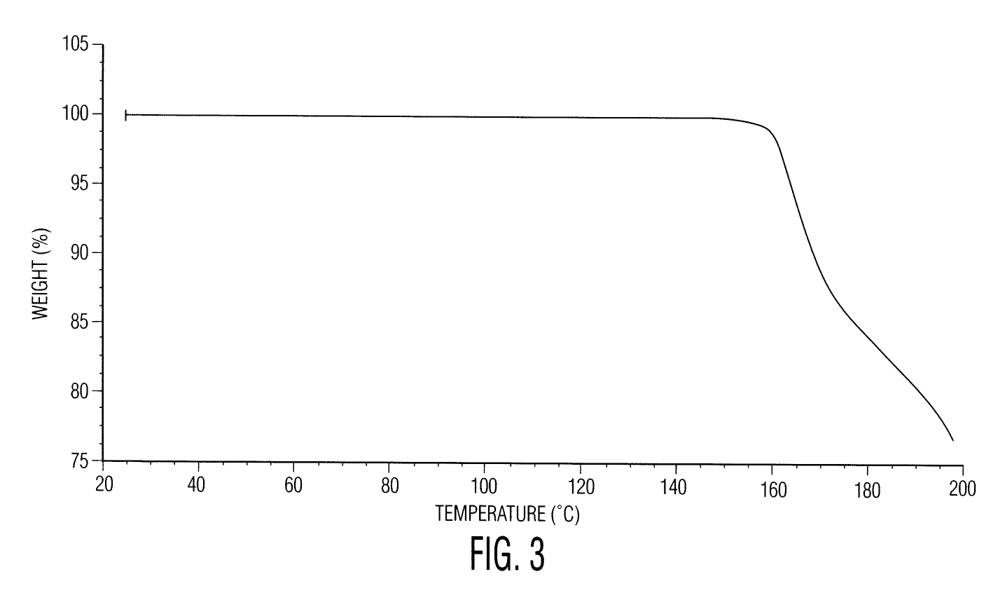

Armodafinil Form I as prepared herein may be also characterized by a TGA curve as represented in FIG. 3, corresponding to a weight loss about 0.04%. TGA analysis has been carried out in a TGA Q500 instrument with a ramp of 10° C./minute, up to 250° C.

Particle size distributions of armodafinil particles may be measured with a Jayant Test Siever (Mesh No. 60; mesh opening: 250 μm). Particle size distributions of armodafinil particles may also be measured using equipment such as a Malvern Master Sizer 2000 (helium neon laser source; armodafinil suspended in light liquid paraffin; size range: 0.02 μm to 2000 μm).

Pharmaceutically acceptable excipients for preparing formulations include, but are not limited to: diluents such as starches, pregelatinized starches, lactose, powdered cellulose, microcrystalline cellulose, dicalcium phosphate, tricalcium phosphate, mannitol, sorbitol, and sugar; binders such as acacia, guar gum, tragacanth, gelatin, polyvinylpyrrolidones, hydroxypropyl celluloses, hydroxypropyl methylcelluloses, and pregelatinized starches; disintegrants such as starches, sodium starch glycolate, pregelatinized starches, crospovidones, croscarmellose sodium, and colloidal silicon dioxide; lubricants such as stearic acid, magnesium stearate, and zinc stearate; glidants such as talc and colloidal silicon dioxide; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethyl celluloses, methyl celluloses, various grades of methyl methacrylates, and waxes. Other pharmaceutically acceptable excipients that are of use include, but are not limited to, film formers, plasticizers, colorants, flavoring agents, sweeteners, viscosity enhancers, preservatives, and antioxidants.

Certain specific aspects and embodiments of the present application will be explained in greater detail with reference to the following examples, which are provided by way of illustration only and should not be construed as limiting the scope of the application in any manner.

### Example 1

### Preparation of Armodafinil

STEP A: Preparation of benzhydryl Thioacetic Acud.

Thiourea (4.34 Kg), conc. HCl (6.61 L) and water (30 L) were charged into a reactor and stirred for 10±2.5 minutes. Diphenylmethanol (15.00 Kg) was added and the mass was heated to about 72.5±2.5° C. for about 45 to about 60 minutes. The mass was cooled to about 30±5° C. and the precipitated solid was filtered. The wet cake was washed with water (2×15 L) and dried for about 90 minutes. Solid was dissolved in water (37.50 L) and caustic soda lye (21.45 L) was added to it at a temperature below 35° C. The mixture was stirred for about 4 hours at about 30±5° C. The mixture was heated to about 45±5° C. and chloroacetic acid solution (8.47 Kg in 16.95 L of water) was added. The mixture was heated to about 85±5° C. and stirred for about 90 minutes. The mixture was cooled to 32.5±2.5° C. and water (45 L) was added. The pH was adjusted to about 1 to 2 using conc. HCl (19.5 L) with stirring for about 30 minutes. The solid was filtered and washed with water (15 L). The wet cake was dried at about 30±5° C. under reduced pressure for about 9 hours to afford the title compound (yield: 91.9%).

STEP B: Preparation of (±)-Benzhydrylsulfinyl Acetic Acid.

Benzhydrylthioacetic acid (19.00 Kg) and glacial acetic acid (57 L) were charged into a reactor and the mass was cooled to about 20±3° C. Hydrogen peroxide (˜50%; 5.80 L) was slowly added to the mass at about 20±3° C. and stirred for about 4 hours. After addition of water (104.50 L) to the mass, it was heated to about 30±5° C. and stirred for about 45 minutes. The solid was filtered and washed with water (38 L). The wet cake was dried at about 42.5±2.5° C. under reduced pressure for about 6 hours. Ethyl acetate (95 L) was added to the dry solid material and heated at about 67.5±2.5° C. for about 45 minutes. The mass was cooled to about 30±5° C. and stirred for about 45 minutes. The solid was filtered and washed with ethyl acetate (19 L). The wet cake was dried below 45° C. under reduced pressure for about 7 hours to afford the title compound (yield: 80.23%).

STEP C: Preparation of (−)-α-Methylvenzylamine-(−)-Benzhydrylsulfinyl Acetic Acid.

(±)-Benzhydrylsulfinyl acetic acid (16.00 Kg) and water (160 L) were charged into a reactor and heated to about 42.5±2.5° C. L-(−)-α-methylbenzylamine (7.90 L) was added and the reaction mass was heated to about 77.5±2.5° C. over about 10 minutes. The mass was cooled to about 60±5° C. and stirred for about 30 minutes. The mass was further cooled to about 45±3° C. and stirred for about 60 minutes. The solid was filtered and washed with water (16 L). Water (120 L) was added to the wet solid, heated to about 87.5±2.5° C. and stirred for about 15 minutes. The mass was cooled to about 45±3° C. and stirred for about 30 minutes. The solid was filtered and washed with water (16 L). The wet cake was dried at about 47.5±2.5° C. under reduced pressure for about 7 hours to afford the title compound (yield: 68.75%).

STEP D: Preparation of (−)-Benzhydrylsulfinyl Acetic Acid.

(−)-α-methylbenzylamine (−)-benzhydrylsulfinyl acetic acid (8.00 Kg) and water (40 L) were charged into a reactor and heated to about 30±5° C. for about 10 minutes. pH of the mass was adjusted to below 2 using conc. HCl (2.5 L) and it was stirred for about 45 minutes. The solid was filtered and washed with water (6 L). The wet cake was dried at about 45±5° C. under reduced pressure for about 11½ hours to afford the title compound (yield: 89.67%).

STEP E: Preparation of Methyl (−)-Benzhydrylsulfinyl Acetate.

(−)-Benzhydrylsulfinyl acetic acid (5.92 Kg), potassium carbonate (2.98 Kg) and acetone (59.2 L) were charged into a reactor and stirred for about 10 minutes. Dimethylsulphate (3.07 L) was added and the mass was stirred at about 30±5° C. for about 7½ hours. Solvent was distilled completely below 40° C. under reduced pressure. The residue was cooled to about 30±5° C. Water (118.4 L) was added and stirred at about 30±5° C. for about 45 minutes. The solid was filtered and washed with water (11.84 L). The wet cake was dried at about 42.5±2.5° C. under reduced pressure for about 4 hours to afford the title compound (yield: 89.53%).

STEP F: Preparation of Armodafinil.

Methyl (−)-benzhydrylsulfinyl acetate (3 Kg) and methanol (36 L) were charged into a reactor and cooled to about 15±5° C. Ammonia gas was passed through the mass under pressure (1 -2 Kg/cm²) at about 15° C. and stirred for about 3 hours, 15 minutes. The mass was heated to about 30±5° C. Solvent was distilled completely below 40° C. under reduced pressure. Methanol (12 L) and 2-butanol (48 L) were added to the residue and heated to about 67.5±2.5° C. The mass was filtered and 2-butanol (9 L) was added to the filtrate under a nitrogen atmosphere. The mass was cooled to about 7.5±2.5° C. and stirred for about 25 minutes. The solid was filtered and washed with chilled 2-butanol (3 L). The wet cake was dried at about 45±5° C. under reduced pressure for about 4 hours to afford the title compound (yield: 70.87%).

### Example 2

### Preparation of Armodafinil Form I.

Armodafinil (1.35 Kg) was dissolved in 2-butanol (22.95 L), heated to about 65±3° C. and stirred for about 15 minutes. The mass was filtered and 2-butanol (4.05 L) was added at about 62.5±2.5° C. The mass was cooled to about 58±2° C. and seed crystals (40.5 g) were added and stirred. The mass was cooled to about 50±2° C. in about 45 minutes and stirred for about 20 minutes. The mass was cooled to about 40±2° C. in about 75 minutes and stirred for about 30 minutes. The mass was cooled to about 30±2° C. in about 45 minutes and stirred for about 5 minutes. The reaction mass was further cooled to about 2.5±2.5° C. in about 50 minutes and stirred for about 20 minutes. The solid was filtered and washed with chilled 2-butanol (2.70 L). The wet cake was dried at about 57.5±2.5° C. under reduced pressure for about 6 hours to afford the title compound (yield: 81.48%, HPLC purity: 99.97%).

### Example 3

### Preparation of Armodafinil Form I.

Armodafinil (10 g) was dissolved in 2-butanol (200 mL) and heated at about 65±3° C. and stirred for about 45 minutes. The mass was filtered and cooled to about 58±2° C. Seed crystals (300 mg) were added to the solution and stirred for about 30 minutes. The mass was cooled to about 50±2° C. in about 20 minutes and stirred for about 30 minutes. The mass was cooled to about 40±2° C. in about 20 minutes and stirred for about 30 minutes. The mass was cooled to about 30±2° C. in about 20 minutes and stirred for about 30 minutes. The mass was further cooled to about 2.5±2.5° C. in about 60 minutes and stirred for about 30 minutes. The solid was filtered and washed with chilled 2-butanol (2 L). The wet cake was dried at about 57.5±2.5° C. under reduced pressure for about 6 hours to afford the title compound (yield: 86%).

Armodafinil was passed through a sieve having No. 60 mesh (as defined in United States Pharmacopoeia). The coarse particles were kept aside and the fine particles were subjected to micronization by air jet milling (injecting pressure: 0 to 5 kg/cm²; milling pressure: 0 to 5 kg/cm²). 38.95% of armodafinil particles have sizes greater than 250 μm. 61.05% of armodafinil particles have sizes less than 250 μm (D₁₀: 0.697 μm; D₅₀: 13.259 μm; D₉₀: 29.709 μm).

### Example 4

### Preparation of Armodafinil Form I.

Armodafinil (1 g) was dissolved in 2-butanol (40 mL) and heated to about 55±5° C. for about 20 minutes. The mass was filtered and cooled to about 30±5° C. The solution was added to n-heptane (400 mL) and stirred for about 30 minutes. The mass was cooled to about 2.5±2.5° C. in about 15 minutes and stirred for about 30 minutes. The solid was filtered and dried at about 57.5±2.5° C. under reduced pressure for about 5 hours to afford the title compound (yield: 80%).

### Example 5

### Preparation of Armodafinil Form I.

Armodafinil (5 g) was dissolved in N-methylpyrrolidine (7 mL) and heated to about 70±5° C. for about 15 minutes. The mass was filtered and cooled to about 30±5° C. Methyl t-butyl ether (105 mL) was added to the solution. The mass was cooled to about 2.5±2.5° C. in about 15 minutes and stirred for about 2 hours. The solid was filtered and dried at about 80±2.5° C. under reduced pressure for about 5 hours to afford the title compound (yield: 84%).

1. A process for the preparation of armodafinil Form I, comprising:
a) providing a solution of armodafinil in a suitable solvent;
b) precipitating a solid; and
c) optionally, drying the solid.

2. The process of claim 1, wherein a solvent comprises: a C₁-C₄ alcohol; a C₂-C₆ ketone; a halogenated hydrocarbon; an ether; a hydrocarbon; a nitrile; an aprotic polar solvent; a carboxylic acid; nitromethane; ethylene glycol; or a mixture of two or more thereof.

3. The process of claim 1, wherein a solvent comprises methanol, ethanol, 2-butanol, acetone, methyl ethyl ketone, dichloromethane, chloroform, N,N-dimethylformamide, N,N-dimethylacetamide, dimethylsulphoxide, N-methylpyrrolidone, tetrahydrofuran, 1,4-dioxane, acetonitrile, formic acid, acetic acid, nitromethane, ethylene glycol, or a mixture of two or more thereof.

4. The process of claim 1, wherein a solvent comprises 2-butanol or N-methylpyrrolidone.

5. The process of claim 1, wherein a solvent comprises 2-butanol and precipitating in b) is promoted by cooling a solution.

6. The process of claim 1, wherein precipitating in b) is promoted by cooling a solution.

7. The process of claim 1, wherein precipitating in b) is promoted by adding seed crystals of armodafinil Form I and cooling a solution.

8. The process of claim 1, wherein precipitating in b) is promoted by combining an anti-solvent with the solution of a).

9. The process of claim 8, wherein an anti-solvent comprises water, isopropyl alcohol, n-butanol, methyl isobutyl ketone, toluene, a xylene, ethyl acetate, diethyl ether, diisopropyl ether, methyl t-butyl ether, anisole, n-pentane, hexanes, n-heptane, cyclohexane, or a mixture of any two or more thereof.

10. The process of claim 1, wherein a solvent comprises 2-butanol and precipitating in b) is prompted by combining a solution with an anti-solvent comprising n-heptane.

11. The process of claim 1, wherein a solvent comprises 2-butanol and precipitating in b) is prompted by combining a solution with an anti-solvent comprising methyl t-butyl ether.

12. A pharmaceutical composition prepared using:
armodafinil particles, about 30% or more by weight thereof having sizes greater than about 250 μm, and about 70% or less by weight thereof having sizes less than about 250 μm,
wherein, of the particles having sizes less than about 250 μm, about 50% have sizes less than about 50 μm.

13. The pharmaceutical composition of claim 9, wherein armodafinil particles comprise armodafinil Form I.
